(19) United States
(12) Patent Application Publication
(10) Pub. No.: US 2024/0268433 A1
(43) Pub. Date: Aug. 15, 2024

(54) Nutritional Compositions for Preserving Muscle Mass

(71) Applicant: Dutch Medical Food B.V., Venlo (NL)

(72) Inventors: Rudolf Leonardus Lodewijk Smeets, Amsterdam (NL); Robert Johan Joseph Hageman, Wageningen (NL)

(21) Appl. No.: 18/560,566

(22) PCT Filed: May 4, 2022
(86) PCT No.: PCT/NL2022/050244

§ 371 Date: Nov. 13, 2023

(30) Foreign Application Priority Data
May 14, 2021 (EP) 21173932.1

(51) Int. Cl. A23L 33/175; A23L 33/12; A23L 33/19; A61K 38/01; A61P 21/06

(52) U.S. Cl. CPC A23L 33/175; A23L 33/12; A23L 33/19; A61K 38/018; A61P 21/06

(57) ABSTRACT

The invention relates to a nutritional composition, comprising, per 100 kcal, —about 5 to 25.0 g of proteinaceous matter, of which —about 5 to 24.9 g of proteinaceous matter derived from milk; wherein —the serine content is at least about 7 wt. %, based on the weight of the proteinaceous matter, and wherein —the glycine content of the nutritional composition is between 5 and 30 wt. %, preferably between 5 and 20 wt. %, more preferably between 5 and 15 wt. % based on the weight of the proteinaceous matter.

The invention relates to a nutritional composition, to a nutritional composition for use in as a medicament and to a nutritional composition in a method of treatment by therapy, in particular a treatment involving preserving or increasing muscle mass. More in particular the invention relates to a nutritional composition for use in the treatment or prevention of sarcopenia.

Sarcopenia is a medical condition that is characterized by a progressive loss of skeletal muscle mass, strength and function. Skeletal muscle mass is regulated by the physiological opposing processes of muscle protein synthesis and breakdown. These processes are, among others, affected by anabolic stimuli such as food intake and physical activity. For example, dietary protein and/or amino acid intake can stimulate muscle protein synthesis and inhibit muscle breakdown, causing a net gain of skeletal muscle mass.

In the sarcopenic population, a reduced response to such anabolic stimuli is observed, also referred to as ‘anabolic resistance’. Consequently, the equilibrium between muscle catabolism (break down) and muscle anabolism (build up) is disturbed, resulting in a higher catabolic than anabolic rate. As a consequence, a net reduction of muscle mass and a decrease of muscle function, including not only muscle strength, but also speed, endurance and control of the body is observed. This decrease in muscle mass and function may lead to disability, morbidity and ultimately mortality of the subject suffering from sarcopenia. (Wall et al. 2015. PloS ONE 10(11): e0140903; Tessier et al. 2018. Nutrients 10:1099).

Although the term sarcopenia was first used to indicate the age-related loss of muscle mass, nowadays, besides aging, also chronic diseases, infectious diseases, a physical inactive lifestyle, loss of mobility, and malnutrition are recognized as causes of sarcopenia.

Subjects identified as at risk can be diagnosed as sarcopenic, when poor muscle function or muscle mass, e.g., grip strength or walking speed or low fat-free mass is observed. As an initial tool for screening subjects that are at risk of having sarcopenia, a questionnaire may be used, such as the SARC-F test including questions on muscle strength, walking, chair rise, stairs climbing and falls. In addition, calf circumference may be measured to improve sensitivity of the SARC-F test. (Malstrom et al. 2013. JAMDA, 14:531-532; Morley, 2021. J. Nutr Health Aging. 25(3): 278-280)

Additional practical tests have been developed as an aid to diagnose sarcopenia, amongst others by the European working group on sarcopenia in older people (EWGSOP2). The cut off points recommended by the EWGSOP2 are summarized in Table 1 (Crutz-Jentoft, et al. 2019. Age and Ageing, 48: 16-31).

TABLE 1
EWGSOP2 sarcopenia cut-off points

| | | |
|---|---|---|
| | Cut-off points for | |
| Test | men | Cut-off points for women |

EWGSOP2 Cut off points for low strength

| | | | | |
|---|---|---|---|---|
| Grip strength | <27 | kg | <16 | kg |
| Chair stand | >15 | s for five rises | | |

EWGSOP2 Cut off points for low performance

| | | |
|---|---|---|
| Gait speed | ≤0.8 | m/s |

| Timed-Up and Go test | ≥20 s |
|---|---|
| (TUG) | |
| Short physical | test score of ≤8 points score |
| performance battery | |
| (SPPB) | |

| | |
|---|---|
| 400 m walk in | Non-completion or >6 min for completion |

EWGSOP2 Cut off points for low muscle quantity

| | | | | |
|---|---|---|---|---|
| Appendicular skeletal | <20 | kg | <15 | kg |
| muscle mass (ASM) | | | | |
| ASM/heigth² | <7.0 | kg/m² | <5.5 | kg/m² |

Current strategies aiming to prevent or treat sarcopenia typically involve increasing anabolic triggers to stimulate muscle protein synthesis, whilst inhibiting muscle catabolism.

One such anabolic trigger is by increasing the daily protein intake of the subject suffering from or at risk of developing sarcopenia. Therefore, in order to stimulate muscle protein synthesis and overcome anabolic resistance, current nutritional recommendations suggest intake of 1.0-1.2 g protein/kg body weight per day for healthy at-risk elderly, and between 1.2-1.5 g/kg/day for malnourished elderly. This is markedly higher than the 0.8 g protein/kg body weight/day recommended for healthy adults of younger age (Deutz et al. 2014. Clin. Nutr. 33(6):929-936).

Another anabolic trigger for stimulating muscle protein synthesis is by physical exercise. In a study of nursing home residents aged 90 or more, high-intensity resistance training appeared to be beneficial for muscle mass increase, strength and walking speed. Although further research is required, studies suggest that resistance exercise supports muscle mass increase by means of increased insulin sensitivity for both improved glucose utilization and enhanced myofibrillar protein synthesis. (Makanae et al. 2015. J. Nutr. Sci Vitaminol. 61: S125-S127)

Taken together, best results were obtained when using a combination of physical (resistance) exercise and nutritional management. However, this approach was found to be associated with a number of disadvantages.

First of all, it was found that only a small benefit was observed in muscle function and muscle mass, which was not regarded sufficient to appropriately address or prevent the symptoms associated with sarcopenia.

Secondly, in order to obtain a clinically relevant result, high intensity resistance exercise was found to be a key aspect of the treatment or prevention. However, subjects suffering from or at risk of developing sarcopenia are often physically uncapable to perform physical exercise for the time required to prevent sarcopenia or reduce loss of muscle mass, strength or function.

Thirdly, it is essential to develop an accurate exercise program that is both effective for preserving muscle mass and safe for the subject enrolled in the program. Typically, in order to guarantee both aspects, the exercise program is developed and supervised by health care professionals and tailored to the subjects enrolled therein. It is therefore complex to implement physical exercise into the treatment program for sarcopenia in an efficient and effective manner.

Accordingly, there is a need for other nutritional compositions that are effective in maintaining and/or increasing muscle mass and/or function and/or reducing muscle decline in a subject, in particular without requiring that the subject is enrolled in a physical exercise program.

Therefore, the invention relates to a nutritional composition, comprising, per about 100 kcal,
about 5 to 25.0 g of proteinaceous matter, of which
about 5 to 24.9 g of milk proteins selected from the group consisting of whey protein, casein protein or a combination thereof; and wherein
the serine content is at least 7 wt. %, based on the weight of the proteinaceous matter.

The invention further relates to the nutritional composition according to the invention for use in a method of treatment by therapy, in particular for use in maintaining or increasing muscle mass and/or maintaining or increasing muscle function and/or reducing loss of muscle mass and/or reducing loss of muscle function of a human in need thereof; in particular, the nutritional composition according to the invention is useful for use in prevention or treatment of sarcopenia.

The invention further relates to the nutritional composition according to the invention for use in a medical treatment of (or as a medicament in the treatment of) a sleeping disorder.

The invention further relates to the nutritional composition according to the invention for use in a medical treatment of (or as a medicament in the treatment of), which use comprises increasing the average daily proteinaceous matter intake of a person in need thereof to a value of at least 0.8 g/kg bodyweight or maintain at a value of at least 0.8 g/kg bodyweight.

The invention further relates to a use of the nutritional composition according to the invention for improving sleep quality as determined by the Pittsburgh Sleep Quality Index. Such use can in particular be a non-medical use.

The invention further relates to a use of the nutritional composition according to the invention for increasing the average daily proteinaceous matter intake of a person in need thereof to a value of about 0.8-1.0 g/kg bodyweight. Such use can in particular be a non-medical use.

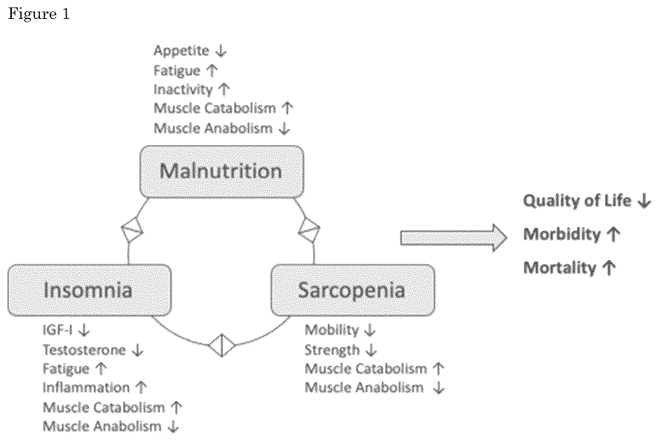

FIG. 1 schematically shows the mutual dependency between biological processes underlying sarcopenia, insomnia and malnutrition.

FIG. 2 shows first results of a clinical trial regarding the effect of a composition according to the invention on sleeping quality.

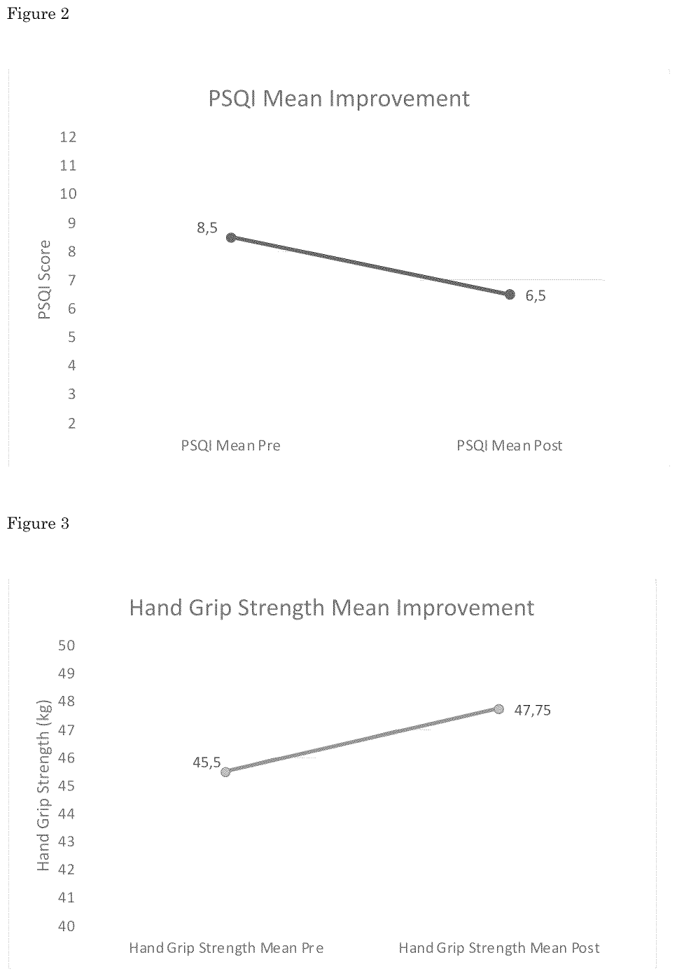

FIG. 3 shows first results of a clinical trial regarding the effect of a composition according to the invention on a muscle function (hand grip strength).

The term “or” as used herein is defined as “and/or” unless specified otherwise.

The term “a” or “an” as used herein is defined as “at least one” unless specified otherwise.

When referring to a noun (e.g. a compound, an additive, etc.) in the singular, the plural is meant to be included.

The term ‘essentially free’ is generally used herein to indicate that a substance is not present (below the detection limit achievable with analytical technology as available on the effective filing date) or present in such a low amount that it does not significantly affect the property of the product that is essentially free of said substance.

In the context of this application, the term ‘about’ means generally a deviation of 15% or less from the given value, in particular a deviation of 10% or less, more in particular a deviation of 5%, 4%, 3%, 2%, 1%, 0.5% or less. ‘Proteinaceous matter’ as used herein refers to a protein or any part of a protein, such as non-hydrolyzed protein, native protein, hydrolyzed protein, peptides, such as oligopeptides and free amino acids. ‘Oligopeptides’ as used herein refers to a peptide comprising two to fifty amino acids. The total content of proteinaceous matter is determinable with the Kjehldahl method as known in the art.

‘Proteinaceous matter derived from milk’ as used herein refers to any protein, mixture of proteins, fraction or part thereof, such as hydrolysates, peptides, or amino acids that is derived from milk from a mammal.

In the context of the present invention, whey protein may refer to the complete protein fraction obtainable from whey or to any fraction or hydrolysate thereof. As is generally known ‘whey protein’ are proteins that remain in the liquid fraction that is obtained after acidification and curdling of milk. Typically, whey protein comprises one or more of the proteins β-lactoglobulin, α-lactalbumin, serum albumin, immunoglobulins, lactoferrin and transferrin, or any part thereof such as a hydrolyzed form thereof.

As is known to a skilled person, ‘casein proteins’ form a group of proteins including acid casein, rennet casein, caseinate, micellar casein, fractions thereof and hydrolysates thereof. As is generally known, casein protein is a supramolecular association of individual casein subunits: αS1-casein, αS2-casein, β-casein, and k-casein. These fractions are organized within, a micellar structure according to a balance of interactions involving their hydrophobic and hydrophilic groups. The casein micelle is held together by colloidal calcium phosphate.

“Rennet casein” is casein obtained by enzymatic precipitation, as described in Walstra, P. et al., Dairy Science and Technology, CRC Press, 2006, pages 538 and 539.

“Acid casein” typically refers to a casein obtained by acid precipitation of casein, typically by acidifying skim milk to the isoelectric point of casein (pH 4.6-4.7).

‘Caseinate’ refers to a non-micellar protein derived from casein, obtainable by acid precipitation from a liquid containing solubilized casein (casein micelles) such as milk, and subsequent neutralization with a base, such as a hydroxide, e.g. NaOH, KOH, Mg(OH)₂, Ca(OH)₂, NH₄OH or a basic salt, e.g. CaCO₃, Na₂CO₃ or K₂CO₃ and mixtures thereof. Like other forms of casein, caseinate is composed of a mixture of four major casein types (alpha S1, alpha S2, beta and kappa casein). Typically, micellar casein contains calcium and phosphate (so-called calcium phosphate nanoclusters) bound to the protein structure, stabilizing the micellar structure.

If an amino acid comprises multiple stereoisomers, with the term ‘amino acid’, e.g. ‘serine’ as used herein is meant the L-amino acid, e.g. L-serine, in any physiologically acceptable form, such as in bound form, in particular in a protein or peptide, or in free form, in particular as free amino acid or salt thereof or physiologically acceptable derivative thereof. With the term ‘glycine’ as used herein, is meant glycine in any possible form, such as in bound form in particular in a protein or peptide, or in a free form in particular as free amino acid or salt thereof or physiologically acceptable derivative thereof.

In the context of the present invention, the term ‘elderly’ is meant to refer to a human, having an age of 50 years or more, preferably 60 years or more, 65 years or more, 70 years or more, or 75 years or more.

In the context of the present invention, the term ‘sarcopenia’ refers to a condition associated with involuntary loss of muscle mass, muscle function and/or strength. Sarcopenia may be diagnosed by a trained physician, for example using the EWGSOP2 criteria (Crutz-Jentoft, et al. 2019. Age and Ageing, 48: 16-31). In the context of the present invention, a subject is suffering from sarcopenia if it fulfills one or more, preferably all of the criteria from Table 1.

The term ‘therapy’ in the context of the present invention, refers to the prevention, medical treatment or cure of a medical condition or disease, including alleviating or relieving one or more symptoms associated with said condition or disease.

### Nutritional Composition

Current strategies towards treatment of prevention of sarcopenia focus on increasing the daily dietary protein intake in combination with physical exercise to stimulate muscle protein synthesis, thereby maintaining or increasing muscle mass, strength and/or function in a subject in need thereof.

Recently, a correlation between sarcopenia and sleep quality was found, indicating that a higher prevalence of sarcopenia was present in subjects having inadequate sleep, compared to control subjects (Rubio-Arias et al. 2019. J. Clin. Mol, 8: 2156). The authors suggest that in subjects suffering from sleep deprivation the balance between anabolic and catabolic hormones is disturbed, characterized by elevated cortisol levels and reduced levels of IGF-1, respectively catabolic and anabolic hormones. Hence, suggesting that insufficient sleep promotes muscle breakdown during the night and thereby contributes to a decrease in skeletal muscle mass and function.

The inventors realized that there appears to be a strong mutual dependence between sleeping problems, malnutrition and sarcopenia, as visualized in FIG. 1. As shown in FIG. 1, each of these processes is characterized by a decrease in muscle anabolism and an increase in muscle catabolism, thus resulting in a net reduction of muscle mass. Hence, the efficient targeting of all these three aspects simultaneously, may provide an efficient tool towards preserving muscle mass and/or function and thus in preventing or treating sarcopenia.

Accordingly, the invention relates to a nutritional composition comprising, about 5 to 25.0 g per 100 kcal of proteinaceous matter, of which about 5 to 24.9 g per 100 kcal of proteinaceous matter is derived from milk; wherein
the serine content of the nutritional composition is at least about 7 wt. %, based on the weight of the proteinaceous matter; and wherein
the glycine content of the nutritional composition is between 5 and 30 wt. %, preferably between 5 and 20 wt. %, more preferably between 5 and 15 wt. % based on the weight of the proteinaceous matter. The nutritional composition according to the invention is in particular suitable for a use as described in the claims or elsewhere in the present description.

The nutritional composition according to the invention is particularly suitable for preserving or increasing muscle mass during sleep. Without wishing to be bound by any theory, it is believed that the nutritional composition provides an anabolic trigger in the form of proteinaceous matter that supports muscle anabolism and inhibits muscle catabolism. In addition, the nutritional composition provides components that improve sleep quality, reduces inflammation and improves the nourishment in a subject in need thereof. By improving sleep and reducing inflammation, muscle catabolism may be further inhibited while muscle anabolism is further increased. Hence, a synergistic effect in maintaining muscle mass and/or function may be obtained by simultaneously addressing the causes underlying malnourishment, sarcopenia and sleeping abnormalities.

Therefore, although the nutritional composition according to the invention may be consumed at any time of the day by a subject in need thereof, the nutritional composition is particularly suitable for administration prior to sleep, preferably about 1 hour or less prior to sleep, more preferably 45 minutes or less prior to sleep, in particular 1-30 minutes prior to sleep.

Studies support that proteinaceous matter ingested in elderly men before sleep was properly digested and absorbed throughout the night, thereby providing amino acid precursors for muscle protein synthesis during sleep (Kouw et al. 2017. J Nutr 147:2252-61). Hence, by administering a nutritional composition before sleep, loss of muscle mass taking place overnight, may be prevented or reduced and muscle protein synthesis stimulated, thereby contributing to maintaining or increasing muscle mass and/or maintaining or increasing muscle function and/or reducing loss of muscle mass and/or reducing loss of muscle function.

The nutritional composition according to the invention comprises about 5 to 25.0 g per 100 kcal of proteinaceous matter, preferably about 7 to about 20 g per 100 kcal, more preferably about 8 to about 17 g per 100 kcal, in particular between about 10 to about 15 g of proteinaceous matter per 100 kcal.

In terms of energy content, the nutritional composition according to the invention, preferably comprises (per 100 kcal) about 25-100 en % of proteinaceous matter, more preferably about 30 to about 90 en %, about 35 to about 80 en %, about 40 to about 70 en %, in particular about between 40 and 50 en % of the total nutritional composition. As the skilled person is aware, the energy content of a nutritional composition may be determined based on the energy component of the individual components. The energy content of a nutritional composition or a fraction thereof, may be experimentally determined using a calorimeter, as is known in the art. Typically in a calorimetry experiment, a nutritional composition is burned and the released energy is used to heat a known quantity of water. The temperature change (ΔT) of the water may be used to determine the amount of energy in the food. However, the energy content of a nutritional composition may also be calculated, by using average energy contents of fats (9 kcal/g), digestible carbohydrates (4 kcal/g) and proteinaceous matter (4 kcal/g).

Essential amino acids, as is known to the skilled person, are amino acids that are not synthesized in sufficient amounts by the human body and therefore have to be provided by nutritional intake. The group of essential amino acids consists of phenylalanine (Phe), valine (Val), threonine (Thr), tryptophan (Trp), methionine (Met), leucine (Leu), isoleucine (Ile), lysine (Lys) and histidine (His). Essential amino acids play an essential role in the process of muscle protein synthesis. Of these amino acids, leucine appears to be most potent, by stimulation of the mammalian target of rapamycin (mTOR) pathway.

The nutritional composition according to the invention preferably comprises at least 20 wt. %, more preferably at least 25 wt. %, in particular at least 30 wt. % of essential amino acids, based on total proteinaceous matter. Serine and glycine are not essential amino acids. Proteinaceous matter from milk typically also comprises further non-essential amino acids, as is generally known. The maximum content of essential amino acids in a composition according to the invention can be determined by the skilled person, based on the total content of the non-essential amino acids. Usually, the total essential amino acid content is about 84 wt. % or less wt. %, in particular about 60 wt. % or less, more in particular about 55 wt. % or less, even more in particular about 50 wt. % or less, based on total proteinaceous matter. Proteins having a high abundance of essential amino acids, such as whey proteins and casein are generally known. If desired, the compositions may be supplemented with one or more essential amino acids, although good results have been achieved without adding essential amino acids in a free form.

Milk proteins, in particular whey proteins, are rich in branched chain amino acids, such as isoleucine, valine and leucine (typically over 5 wt. % of Ile, over 4 wt. % of Val and over 10 wt. % of Leu is present in whey protein from cow milk).

Hence, preferably, the nutritional composition according to the invention, comprises at least 5 wt. % of leucine, more preferably at least 6 wt. %, at least 6.5 wt. %, in particular at least 7 wt. % of leucine, based on the weight of the proteinaceous matter. Usually, the leucine content is about 20 wt. % or less, preferably 12 wt. % or less, in particular 11 wt. % or less, based on total proteinaceous matter.

Hence, preferably, the nutritional composition according to the invention, comprises at least 2 wt. % of isoleucine, more preferably at least 3 wt. %, at least 4 wt. %, in particular at least 5 wt. % of isoleucine, based on the weight of the proteinaceous matter. Usually, the isoleucine content is about 10 wt. % or less, preferably 7 wt. % or less, in particular 6 wt. % or less, based on total proteinaceous matter.

Hence, preferably, the nutritional composition according to the invention, comprises at least 2 wt. % of valine, more preferably at least 3 wt. %, at least 3.5 wt. %, in particular at least 4 wt. % of valine, based on the weight of the proteinaceous matter. Usually, the valine content is about 10 wt. % or less, preferably 7 wt. % or less, based on total proteinaceous matter.

Optionally, a part of the leucine, isoleucine and/or valine is in the form of free leucine, isoleucine and/or valine or salts thereof respectively. However, in an advantageous embodiment, the nutritional composition is essentially free of free leucine, free isoleucine and/or free valine or salts thereof.

In an embodiment, the nutritional composition according to the invention comprises about 5 to 24.5 g proteinaceous matter derived from milk, per 100 kcal, preferably about 5.5 to about 20 g, about 6 to about 15 g, more preferably about 7 to about 10 g, e.g. about 7.5 g or about 9.9 g of proteinaceous matter derived from milk per 100 kcal. Said proteinaceous matter derived from milk may be derived from mammalian milk including milk derived from humans, cows, goat, sheep, camels, horses, donkeys and buffalo. Preferably, the proteinaceous matter derived from milk is of bovine origin.

Proteinaceous matter derived from milk advantageously contains about 30 to 45% of essential amino acids, based on the protein fraction. The content of essential amino acids in milk proteins is significantly higher than in plant-based proteins, which typically comprise about 24-28% of essential amino acids (Gorissen et al. 2018. Amino Acids 50:1685-1695).

Preferably, the nutritional composition according to the invention comprises about 30 to about 99.5 wt. % of proteinaceous matter derived from milk, based on the weight of the proteinaceous matter, more preferably about 40 to about 90 wt. %, even more preferably about 50 to about 75 wt. %, in particular about 60 to about 65 wt. % of proteinaceous matter derived from milk, based on the weight of the proteinaceous matter.

In the nutritional composition according to the invention, the proteinaceous matter derived from milk preferably comprises whey protein. Whey protein contains all essential amino acids. Further, whey protein are considered “fast” proteins referring to the rate of digestion by proteolytic enzymes in the body, thereby allowing a quick release of amino acids into the circulation.

In the context of the present invention, any source of whey protein or fraction thereof, may be used for the preparation of a nutritional composition according to the invention. For instance a whey protein concentrate (WPC), in particular a whey protein isolate (WPI) may be used.

In particular, sweet whey, obtained as a by-product in the manufacturing of cheese, acid whey, obtained as a by-product in the manufacturing of acid casein, native whey, obtained by milk microfiltration or rennet whey, obtained as a by-product in the manufacturing of rennet casein, may be used alone or in combination as source of whey proteins. In particular, whey derived from sweet whey may comprise glycomacroprotein (GMP), a casein-related non-globular protein, which is also soluble at a pH at which the whey proteins are soluble and hence, difficult to separate therefrom.

Whey protein concentrate is a fraction of whey proteins typically obtained by membrane filtration. Whey protein concentrate may comprise, apart from protein, also fat, minerals and/or lactose. A whey protein concentrate is understood as having a percentage of whey protein between the protein content of untreated whey (about 12 wt %) and the protein content of whey protein isolate (at least 90 wt. %). Preferably, whey protein concentrate comprises about 50 wt. % to about 90 wt. % of whey proteins, more preferably about 60 wt. % to about 85 wt. %, in particular about 70 wt. % to about 80 wt. % of whey protein.

Whey protein isolate primarily comprises whey protein (generally at least 90 wt. %) and may optionally comprise small amounts of fat, lactose and/or minerals. The whey protein, used for preparing a nutritional composition according to the invention, may be used in any form, such as a powder or as a liquid, but is preferably used as a powder.

In the nutritional composition according to the invention, the proteinaceous matter derived from milk preferably comprises casein protein. Casein protein is digested by proteolytic enzymes more slowly than whey protein and is therefore considered a “slow” protein.

In the context of the present invention, any source of casein protein or fraction thereof, may be used in the nutritional composition according to the invention, such as micellar casein, non-micellar casein, micellar caseinate, non-micellar caseinate (including sodium and potassium caseinate), lactic acid casein, mineral acid casein, alpha-casein, beta-casein, kappa-casein, a casein fraction, an alpha-casein fraction, a beta-casein fraction, a kappa-casein fraction, casein treated by ultra high-pressure (UHP) processing, translucent casein or any combination thereof. Further, casein protein may be isolated from milk using any method known in the art.

If micellar casein is used in the nutritional composition according to the invention, it can be provided as a relatively pure ingredient, e.g. as micellar casein isolate (MCI) or micellar casein concentrate (MCC). MCI and MCC are obtained by drying (e.g.) spray drying a micellar solution of casein. As a rule of thumb, MCI generally contains at least about 90 wt. % micellar casein and up to 10 wt. % whey protein. However, it is also possible to use other ingredients providing micellar casein and a higher relative amount of whey protein, such as whole milk protein, skimmed milk (powder), milk protein concentrate (MPC), milk protein isolate (MPI).

In the preparation of the nutritional composition according to the invention, casein may also be provided as a co-precipitate of casein and whey protein, e.g. by heating skim milk to a high temperature and then precipitating the casein/whey protein complex, usually with calcium chloride.

Optionally, the nutritional composition according to the invention comprises caseinate, preferably sodium caseinate.

The nutritional composition according to the invention preferably comprises whey protein and casein protein. If both are present, the ratio whey protein to casein can be chosen in a wide range, usually in the range of 1:20 to 20:1, in particular in the range of 1:9 to 9:1. A combination of whey protein and casein allows an advantageous release of amino acids of time, due to the difference in digestion rates of whey protein and casein protein by proteolytic enzymes.

In a first advantageous embodiment, the ratio whey protein to casein is between 4:1 and 1:4, more preferably between 3:1 and 1:3, even more preferably in a ratio between 2:1 and 1:2, in particular about 1:1.

In particular, good results with respect to improving a muscle parameter, such as muscle strength or muscle mass, have been achieved with a nutritional composition wherein the ratio whey protein to casein is relatively close to the ratio in bovine milk. Thus, in a further advantageous embodiment, the ratio whey protein to casein is in the range of 2:1 to 9:1, more preferably in the range of 3:1 to 8:1, in particular 4:1 to 6:1. A relatively high whey protein content, compared to the casein content may for instance facilitate reconstitution of a powdered composition according to the invention in water. Further, adjusting the ratio whey protein to casein can be used to adjust an organoleptic property, such as taste.

Preferably the nutritional composition comprises about 30 to about 99.5 wt. % of whey protein plus casein protein, based on the weight of the proteinaceous matter fraction, more preferably about 40 to about 90 wt. %, even more preferably about 50 to about 75 wt. % of whey protein plus casein protein, based on the weight of the proteinaceous matter. In a specific embodiment, the nutritional composition comprises about 60 to about 65 wt. % of whey protein and casein protein, based on the weight of the proteinaceous matter.

In particular, good results with respect to improving a muscle parameter, such as muscle strength or muscle mass, have been achieved with a nutritional composition having a content of milk protein selected from the group consisting of casein and whey protein in the range of 60 to 90 wt. %, in particular in the range of about 70-85 wt. %, such as about 78 wt. %, based on the weight of the proteinaceous matter.

In an advantageous embodiment, the nutritional composition comprises about 2.5-5 g of whey protein and about 2.5-5 g of casein protein, preferably about 3.5-4 g of whey protein and about 3.5-4 g of casein protein, per about 100 kcal. Such a mixture provides a high content of essential amino acids whilst ensuring a constant release of amino acids over time, due to the difference in digestion rates of whey protein and casein protein by proteolytic enzymes.

In particular, good results have been achieved with a composition having a whey protein content in the range of 5-12 g/100 kcal more preferably 7-10 g/100 kcal, such as about 8.4 g/100 kcal the composition further having a casein content in the range of 1.0-2.0 g/100 kcal, such as about 1.5 g/100 kcal. Such a mixture provides a high content of essential amino acids whilst ensuring a desirable release of amino acids of time, due to the difference in digestion rates of whey protein and casein protein by proteolytic enzymes and has found to be beneficial in particular to improve muscle mass or function in accordance with the invention, for instance in a use wherein the composition is administered shortly before going to sleep.

The nutritional composition according to the invention further optionally comprises about 1 to about 5 g of a plant-based protein, preferably about 1.25 to 3 g, more preferably about 1.5 to 3.5 g of plant-based protein, per 100 kcal. Hence, the content of plant-based protein of the total fraction of proteinaceous matter is preferably around 5 to 70 wt. %, preferably about 10 to 50 wt. %, more preferably about 12 to about 30 wt. %, in particular about 15 to about 25 wt. % of proteinaceous matter.

Plant-based proteins are advantageously derived from plant material and are therefore considered more sustainable than milk proteins, because it does not require the farming of animals, such as livestock. Plant-based protein may further be used in addition to milk proteins to further adjust the release rate of amino acids into the blood stream,

The plant-based protein may be any protein isolated from a plant or plant part, such as a protein isolated from the seeds, roots, leaves, stems, tuber, fruits or flower from a plant. Examples of plant-based proteins are proteins derived from hemp, lupin, oat, corn, rice, pea, potato, wheat, soy, almond, lentils, chickpeas, peanuts, walnuts, quinoa, spirulina, chia and beans. Preferably, the plant based protein is soy protein, almond protein or a combination thereof. In a particularly preferred embodiment the nutritional composition comprises soy protein, in addition to casein and whey protein. An advantageous of including soy protein is the relatively high content of serine and glycine, which is gradually released into the blood stream after intake, as the protein is digested. The soy protein content in such composition is usually 1-60 wt. %, preferably 5-20 wt. %, of the proteinaceous matter. In particular, good results have been achieved with such a nutritional composition having a soy protein content in the range of 6-12 wt. %, such as about 9.5 wt. %, based on the proteinaceous matter.

The content of essential amino acids in soy protein is typically about 27 wt. %. Further, soy protein has a relatively high content of glycine (about 2.7% of the total protein). (Gorissen et al. 2018. Amino Acids 50: 1685-1695). Soy protein has a favorable amino acid profile and therefore complements the amino acid balance of the proteinaceous matter fraction of the nutritional composition according to the invention beneficially.

The serine content of the proteinaceous matter fraction of the nutritional composition according to the invention is at least 7 wt. %, based on the weight of the proteinaceous matter fraction.

Serine is a non-essential amino acid, which can be enzymatically bio-synthesized in the human body in three steps starting from 3-phosphoglycerate, an intermediate from glycolysis, via intermediates 3-phosphohydroxypyruvate and O-phosphoserine.

In two clinical studies evaluating adults between the age of 25 and 59 years, ingestion of serine about 30 minutes before going to sleep, was found to have a beneficial effect on the quality of sleep, both in terms of sleep initiation and nighttime awakenings, resulting in improved feelings of having slept well when waking in the morning (Ito et al. 2014. SpringerPlus 3: 456).

Without wishing to be bound by any theory, it is believed that serine acts as a gamma-aminobutyric acid (GABA) A receptor activator. GABAA is the major inhibitory neurotransmitter in the central nervous system and activation thereof is associated with a sedative and anxiolytic effect. Studies confirmed that administration of picrotoxin, a known GABAA receptor antagonist attenuated the sedative and hypnotic effect of serine in chicken. (Shigemi, K. et al. 2008. Eur J Pharm 599:86-90). Hence, supporting that serine may improve sleep initiation and reduce nighttime awakenings by acting on the GABAA receptor.

Advantageously, studies suggest that, contrary to many known sleep-improving medicines, serine did not appear to suffer from resistance or occurrence of rebound insomnia after discontinuation of ingestion. (Ito et al. 2014. SpringerPlus 3: 456)

According to the invention, serine may be provided by different sources of proteinaceous matter, including but not limited to in bound form as part of intact protein, such as intact milk protein, hydrolyzed protein, as free amino acid or a salt thereof.

The content of serine in milk protein is typically between about 5 and about 6 wt. %. The content of serine in plant proteins is typically between about 2 and 5.5 wt. %. Therefore, in order to provide a nutritional composition comprising at least 7 wt. % of serine, based on the total proteinaceous matter content, the nutritional composition according to the invention comprises a source of proteinaceous matter having a high serine content, preferably free serine.

Preferably, the total serine content of the nutritional composition according to the invention, is at least 7.9 wt. %, at least 8.0 wt. %, at least 8.1 wt. %, at least 8.2 wt. % or at least 8.3 wt. %, e.g. around 8.4 wt. % based on the total proteinaceous matter fraction. Usually, the total serine content of the nutritional composition according to the invention is about 40 wt. % or less, preferably about 30 wt. % or less, about 20 wt. % or less, about 10 wt. % or less, about 9 wt. % or less, more preferably 8.5 wt. % or less, based on the weight of the proteinaceous matter. With such serine contents typically a satisfactory result in preserving muscle mass was observed, while the risk of adverse effects minimized.

According to the invention, the nutritional composition generally comprises up to about 2.5 kcal serine per 10 kcal. The nutritional composition, typically comprises between about 0.25 g and about 2 g of serine per 100 kcal, preferably between about 0.5 g and about 1.5 g of serine, more preferably between about 0.75 g and about 1.25 g of serine, in particular between about 0.9 and 1.1 g of serine, per about 100 kcal. In a specifically preferred embodiment, the nutritional composition comprises between about 0.75 g and about 2.5 g of serine per 100 kcal.

Usually, the nutritional composition according to the invention has a content of free serine or a salt thereof in the range of about 100 to about 1000 mg per 100 kcal, in particular in the range of about 150 to about 750 mg per 100 kcal.

Good results in terms of a positive effect on sleeping behavior and on a muscle parameter, such as muscle strength or muscle mass, have been achieved with a nutritional composition having a content of free serine or salt thereof of about 300 mg/100 kcal or more, more preferably of about 330 to about 600 mg per 100 kcal, in particular of about 350-500 mg per 100 kcal, more in particular of about 365-450 mg per 100 kcal of free serine or a salt thereof.

In a further preferred embodiment, the nutritional composition according to the invention comprises about 150 to about 300 mg per 100 kcal of serine in a free form (free serine or a salt thereof), preferably about 200 to about 250 mg per 100 kcal, more preferably about 225 to about 250 mg per 100 kcal of serine in a free form.

The nutritional composition according to the invention usually has a glycine content of 2 wt. % or more, based on proteinaceous matter. The glycine content of the nutritional composition usually is 30 wt. % or less based on the proteinaceous matter. Preferably, the glycine content is 20 wt. % or less, based on the weight of the proteinaceous matter, more preferably between 5 and 15 wt. %, even more preferably between 8 and 13 wt. %, based on the weight of proteinaceous matter.

Without wishing to be bound by any theory, it is believed that glycine has a positive effect on sleep, by acting on the N-methyl-D-aspartate glutamate receptors and glycine receptors. Studies show that co-administration of glycine with strychnine, a glycine receptor antagonist, inhibited the hypnotic effect of glycine in chicken (Shigemi, 2008. Eur J Pharmacol 59:986-990).

Three clinical studies indicate that ingestion of glycine prior to sleep in female individuals that complain about their sleep quality had a beneficial effect on the subjective quality of sleep (Bennai et al. 2012. J Pharmacol Sci 118: 145-148). In addition, a positive effect of glycine on daytime sleepiness was observed, especially in the morning. The latter may have an additional beneficial effect on the amount of physical exercise a person will do in the morning and with that the anabolic effect during daytime.

Further, glycine is a precursor for a number of metabolites, including creatine, an organic compound which can alter cellular homeostasis thereby protecting the muscle, especially in a condition wherein inflammatory molecules are increased. As substantiated herein above, sleep problems are associated with an increased expression of inflammatory molecules. Hence, it is hypothesized that ingestion of glycine may also have an additional anti-inflammatory component that is beneficial in promoting sleep and thereby beneficial in preserving muscle mass.

Hence, providing additional glycine may have a beneficial effect on sleep and thereby reducing decrease of muscle mass during sleep.

According to the invention, glycine may be provided by different sources of proteinaceous matter, including but not limited to in bound form as part of intact protein, such as intact milk protein, hydrolyzed protein, as free amino acid or a salt thereof.

Glycine is typically present in an amount of about 1 to 1.8% in milk proteins and about 1.5 to 5% of plant-based proteins.

Therefore, in order to provide a nutritional composition comprising at least 2 wt. %, in particular of at least 5 wt. %, of glycine, based on the total proteinaceous matter content, the nutritional composition according to the invention comprises a source of proteinaceous matter having a high glycine content, preferably glycine in a free form (free glycine or salt thereof).

Preferably, the nutritional composition according to the invention comprises about 300 mg to about 2000 mg per 100 kcal of free glycine or a salt thereof, preferably about 1000 to about 1500 mg per 100 kcal of free glycine or a salt thereof.

According to the invention, the nutritional composition preferably comprises between about 0.25 g and about 2.0 g per 100 kcal of total glycine, preferably between about 0.5 g and about 1.5 g per 100 kcal of glycine, more preferably between about 0.75 and about 1.25 g per 100 kcal of glycine, in particular between about 0.9 and about 1.1 g per 100 kcal of total glycine. With such glycine content typically a satisfactory result in preserving muscle mass was observed, while the safety regulations are taken into account and the risk of adverse effects minimized.

In an advantageous embodiment, the nutritional composition according to the invention comprises choline. If present, the composition, preferably comprises about 30 mg to about 150 mg of choline, more preferably about 75 mg to about 125 mg of choline, in particular about 100 mg of choline per 100 kcal. Choline is considered to be advantageous because it can be derivatized into glycinevia inter-organ metabolism of the liver and kidneys.

Preferably, the total content of glycine and serine is at least 1.4 g, preferably at least 1.75 g, more preferably at least 2 g, at least 2.5 g, or at least 3 g per about 100 kcal. Usually, the total content of glycine and serine is less than 12 g/100 kcal, preferably about 9 g/100 kcal or less, more preferably about 6 g/100 kcal or less, e.g. about 3 g/100 kcal or less.

Beta-hydroxy beta-methylbutyrate is a metabolite of leucine. HMB is also known under the names beta-hydroxyisovaleric acid and 3-hydroxyisovaleric acid.

Studies support that HMB promotes muscle protein synthesis. It has been described that the effect of HMB is beneficial for improving muscle mass and muscle function in older people. Specifically, HMB has an antagonistic effect on the protein degradation pathway, thereby inhibiting muscle catabolism. Furthermore, HMB stimulates the mTOR pathway which is an important anabolic pathway in the body, thereby enhancing muscle protein synthesis (Kaczka et al. 2019. J Hum Kin 68:211-222).

Further, studies indicate that HMB promotes the synthesis of growth hormone 1 and IGF-1, both considered anabolic triggers. As substantiated herein above, sleep deprivation is associated with decreasing IGF-1 levels. The addition of HMB in a nutritional composition may thus be beneficial in preventing the reduction of IGIF-1.

Further, HMB acts also directly on the phosphorylation of serine-threonine kinase, an enzyme responsible in the regulation of fundamental cellular processes including proliferation of muscle cells. Kaczka et al. 2019. J Hum Kin 68:211-222).

Still further, it appears that the effect of HMB is not enhanced by physical exercise. This aspect makes HMB an interesting nutritional component for subjects that are not able to perform physical exercise, such as hospitalized subjects, or subjects that are physically incapable of performing exercise. This is confirmed by a study showing that elderly adults exposed to bedrest benefited significantly from HMB supplementation. In this study, HMB was able to attenuate and prevent derangements in skeletal muscle metabolism and mitochondria during bed rest induced muscle atrophy. (Angelus Costa Riela et al. 2021. Ann Nutr Metab:1-7).

According to the invention, the HMB may be free HMB or a HMB salt, in particular calcium HMB, or a combination thereof. Preferably, at least a substantial part of the HMB is calcium HMB, preferably about 50-100 mol. % of the total HMB. Using calcium HMB in a nutritional composition according to the invention is practical from a processing point of view, because calcium HMB is in solid form at conditions wherein it is usually processed. Hence, using calcium HMB in a nutritional composition according to the invention increases flexibility of the process of manufacturing the nutritional composition according to the invention. Further nutritional compositions stored in solid form are typically associated with a longer shelf-life. Hence, using calcium HMB may also positively effect shelf-life of the nutritional composition according to the invention, in a solid from, such as a powder. An advantage of including at least part of the HMB as free HMB is its contribution to a fresh, pleasantly sour taste of the nutritional composition. Such taste is for instance appreciated by persons having difficulties to eat, e.g. after surgery, radiotherapy or chemotherapy.

If present, the total HMB content is generally in the range of 0.5-20 mmol/100 kcal, in particular in the range of 1.0-10 mmol/100 kcal, preferably in the range of about 1.5 to about 9 mmol per 100 kcal, more preferably in the range of about 1.8 to about −5.5 mmol/100 kcal, e.g. about 2.7 to about 3.6 mmol/100 kcal.

Preferably, the nutritional composition according to the invention comprises about 400 mg to about 2500 mg per 100 kcal of calcium beta-hydroxy beta-methylbutyric acid (Ca HMB), preferably about 500 mg to about 1500 mg per 10 kcal, in particular between 750 and 1000 mg of Ca HMB per 100 kcal.

The nutritional composition according to the invention further preferably comprises about 10 to about 50 mg of palmitoylethanolamide (PEA), more preferably about 20 to about 40 mg of PEA, in particular about 25 to about 35 mg of PEA per about 100 kcal. Without wishing to be bound by any theory, PEA is envisaged to have anti-inflammatory properties that may contribute to reducing the inflammatory component associated with loss of muscle mass, muscle function or muscle strength.

The nutritional composition according to the invention further preferably comprises essential vitamins and minerals. The presence of essential vitamins and minerals is advantageous for preventing or treating malnourishment in a subject, which may be an underlying cause of sarcopenia or sleep deprivation, as substantiated herein above.

Preferably, the nutritional composition comprises one or more, preferably at least 4 or more, more preferably at least 6 of, even more preferably at least 12, in particular all of vitamin D, vitamin B12, vitamin B6, Vitamin A, Vitamin K, Vitamin C, folate, thiamin, riboflavin, niacin, pantothenic acid, biotin, vitamin E, sodium, chloride, potassium, calcium, phosphorus, magnesium, iron, zinc, copper, iodine, selenium, manganese, chromium, molybdenum or fluoride in amounts prescribed by commission delegated regulation (EU) 2016/128. In a highly preferred embodiment, the nutritional composition comprises vitamin D, vitamin B12, vitamin B6 and folate. These components are associated with beneficial effects on muscle mass or muscle function.

The nutritional composition according to the invention may further comprise a fat and a carbohydrate. The nutritional composition has a total content of fat plus carbohydrate of about 0-80 en %, preferably between about 10 and about 70 en %, between about 25 and about 60 en %, in particular between about 40 and a 50 en % per serving. The presence of carbohydrates and fats increases the caloric value of the nutritional composition, which is particularly beneficial in subjects that struggle with meeting the required daily caloric intake.

Any source of edible fat is suitable for use in the nutritional composition according to the invention, such as animal fat, such as lard or butter, or a vegetable oil. Examples of vegetable oils include rapeseed oil, sunflower oil, corn oil, soybean oil, coconut oil, palm oil, linseed oil, olive oil.

In practice, it has been found advantageous to provide fat, carbohydrate or both as parts of an ingredient also used for the proteinaceous matter. In particular, casein, whey protein or both may be provided as part of a composition comprising milk fat (e.g. about 0.5 to about 2 g/100 kcal) and/or carbohydrate (such as lactose, glucose, galactose). For instance, WPC (e.g. WPC80) is typically a source of fat and carbohydrate. A further advantage of a milk fat is its positive contribution to an olfactory effect (taste, mouthfeel), compared to vegetable oils and marine oils. Besides, fats from milk can provide some polyunsaturated fatty acids, including omega-3-fatty acids; e.g. in an amount of about 3 wt. % or less respectively about 1 wt. % or less based on total fatty acids.

Polyunsaturated fatty acids are not needed to achieve an effect on muscle or sleeping. The composition may be essentially free thereof or contain only a relatively small amount, such between 0 and 3 wt. % based on total fatty acids of polyunsaturated fatty acids, respectively between 0 and 1 wt. % based on total fatty acids of omega-3 fatty acids. Such composition can be particularly preferred in view of an easier to achieve good consumer compliance, than with compositions having a high omega-3 fatty acid content, typically a liquid oil ingredient is needed.

It is considered that a further positive effect on muscle or sleep is achieved at least in some embodiment, when including (a relatively high amount of) omega-3-fatty acids. Accordingly in a further preferred embodiment, the nutritional composition according to the invention comprises a source of omega-3-fatty acids providing about 33.3 to about 150 mg of an omega-3-fatty acid selected from eicosapentaenoic acid (EPA), docosahexaenoic acid (DHA) and a combination thereof, more preferably about 50 to about 150 mg, more preferably about 75 to about 125 mg. Such a nutritional composition can further contribute to an improvement in sleep and is thereby efficient in preserving muscle mass, a muscle function, or both, during sleep.

Without wishing to be bound by any theory it is believed that omega-3 polyunsaturated fatty acids, including docosahexaenoic acid (DHA) and eicosapentaenoic acid (EPA) play a role in improving sleep quality. Animal studies suggest that dietary deficiency of omega-3-polyunsaturated fatty acids affect sleep regulation including impaired functioning of superchiasmatic nuclei, altered melatonin release and disruption to endocannabinoid signaling. Further, a study of 84 healthy adults suggests that supplementation of DHA or EPA has positive effects on sleep (Patan et al. 2021. Nutrients 13: 248). Hence, supplementation of subjects, in particular elderly subjects, more specifically elderly malnourished subjects or elderly subjects at risk of malnutrition, with an omega-3-fatty acid selected from eicosapentaenoic acid (EPA), docosahexaenoic acid (DHA) and a combination thereof is beneficial in improving sleep and thereby preserving muscle mass during sleep.

Further, EPA/DHA is associated with an anti-inflammatory action which may protect the muscle in case of increased muscle inflammation.

The nutritional composition according to the invention may comprise digestible and/or indigestible carbohydrates. From a dietary perspective, it may be preferred that the nutritional composition is essentially free of simple, or high-glycemic carbohydrates. Simple carbohydrates are carbohydrates comprised of one or two monosaccharides. Simple carbohydrates are typically a fast source of energy, referring to the rate of digestion in the body, compared to complex carbohydrates. Upon digestion of simple carbohydrates the blood sugar level and insulin production are typically increased. Simple carbohydrates are therefore typically also referred to as high glycemic carbohydrates. Increased insulin production has been linked to reduced melatonin production, thereby reducing the sleep quality in a subject.

Examples of simple carbohydrates include glucose, fructose, maltose, sucrose, lactose and galactose.

From a dietary perspective it may be preferred that the nutritional composition according to the invention comprises, based on the dry weight of the nutritional composition comprises less than 5 wt. %, preferably less than 3 wt. %, more preferably less than 2 wt. %, less than 1 wt. %, in particular is essentially free of the carbohydrates glucose, fructose, maltose, sucrose, lactose and galactose.

However, in practice it may be preferred to include a substantial amount of simple carbohydrates, e.g. since they can be present in common sources for proteinaceous matter, such as WPC or commercially available sources of HMB. It has also been found that compositions comprising a relatively high content of carbohydrates remain effective, as illustrated in the Examples. Thus, the composition according to the invention may contain up to about 20 g/100 kcal carbohydrates, in particular 3-15 g/100 kcal, more in particular 5-12 g/100 kcal, e.g. about 10 g/100 kcal carbohydrates.

In a specific preferred embodiment, the invention relates to a nutritional composition, comprising, per 100 kcal
about 7.5 to about 15 g of proteinaceous matter, of which
about 5 to about 10 g of proteinaceous matter derived from milk;
about 750 to about 1250 mg of free glycine or a salt thereof; and
about 200 to about 300 mg of free serine or a salt thereof;
the composition further comprising
about 600 to about 900 mg of calcium beta-hydroxy beta-methylbutyric acid (HMB);
about 75 to 125 mg of an omega-3-fatty acid selected from eicosapentaenoic acid (EPA), docosahexaenoic acid (DHA) and a combination thereof;
about 75 to about 125 mg of choline;
carbohydrates and/or fat.
   In a further specific preferred embodiment, the invention relates to a nutritional composition, comprising,
      60 to 90 wt. % milk protein selected from the group consisting of casein and whey protein,
      2.6-15 wt. %, preferably 3.0-10 wt. % free serine,
      5-15 wt. %, preferably 8.0-12 wt. % free glycine,
      0-15 wt. %, preferably 5.0-10 wt. % soy protein
      all based on weight of the proteinaceous matter

Such composition has been found particularly suitable for improving sleeping pattern in combination with improving a muscle parameter, such as muscle strength or muscle mass. In the nutritional composition of this further specific preferred, the total serine content is usually at least about 7.5 wt. %, preferably 7.5-20 wt. %, in particular 7.5-12 wt. %, more in particular 7.9-10 wt. %, based on total proteinaceous matter; in this nutritional composition, the total glycine content is usually at least about 7.0 wt. %, preferably 8.0-20 wt. %, in particular 9.0-15 wt. %, more in particular 9.0-13 wt. % based on total proteinaceous matter.

The nutritional composition of this further specific preferred embodiment usually further contains HMB and choline. The HMB content in this embodiment is usually in the range of 1.5-10 mmol/100 kcal, preferably in the range of 1.8-5.5 mmol/100 kcal, in particular in the range of 2.7-3.6 mmol/100 kcal. At least a substantial part thereof preferably is calcium HMB. The choline content in this embodiment is usually about 30 mg to about 150 mg per 100 kcal.

The nutritional composition of this further specific preferred embodiment usually further contains fat, preferably milk fat. The fat content, preferably the milk fat content, usually is in the range about 0.5 to about 2 g/100 kcal.

The nutritional composition of this further specific preferred embodiment usually further contains carbohydrate, usually in the range of 1-15 g/100 kcal, in particular about, 3-15 g/100 kcal, more in particular 5-12 g/100 kcal.

The nutritional composition according to the invention is preferably packaged as a serving of about 80 to about 400 kcal, preferably between about 100 and about 300 kcal per serving, more preferably about 150 and 250 kcal per serving, in particular about 180 kcal to about 220 kcal per serving.

The nutritional composition is preferably packaged as a serving of at least 80 kcal, preferably at least 90 kcal, more preferably at least 100 kcal per serving.

Generally, the recommended dietary daily caloric intake is around 2000 kcal a day for women and 2500 kcal for men. However, it was empirically established that many subjects, such as elderly subjects, or subjects suffering from a disease or other medical condition, e.g. a syndrome, struggle to fulfill their daily caloric intake. Failure to meet the daily caloric intake contributes to the subjects getting into a malnourished state, which contributes to developing a medical condition associated with loss of muscle mass and/or function, including sarcopenia. A serving comprising at least 80 kcal may thus beneficially boost the total daily caloric intake of a subject.

Administration of a nutritional composition comprising at least 80 kcal per serving thus advantageously provides already about at least 3 to 4% of the recommended total daily caloric intake, and specifically about 4 to about 12 g of proteinaceous matter. Advantageously, the nutritional composition according to the invention may be administered on top of the regular daily caloric intake, and thus may increase the daily caloric intake, and specifically the intake of proteinaceous matter, of a subject in need thereof.

Preferably, the nutritional composition is packaged as a serving comprising less than 400 kcal, preferably less than 300 kcal, more preferably less than 250 kcal per serving. A serving of about 400 kcal provides about 15% to 20% of the recommended daily caloric intake and thus significantly contributes to increasing the daily requirement of caloric intake. A serving of about 400 kcal furthermore advantageously comprises about 20 to about 60 g of proteinaceous matter.

The nutritional composition according to the invention may have any form or physical condition, for example as a solid, a liquid, a gel, a semi-solid or the like.

Preferably the nutritional composition is formulated as a solid composition, preferably a powder which is preferably capable of being reconstituted in a suitable liquid medium, such as water or aqueous solutions. For example, a serving of 80-400 kcal, preferably 250 kcal, of the nutritional composition according to the invention may be suitable for dissolving in between 50 and 300 ml of water, preferably between 100 ml and 200 ml water. Advantageously, if the nutritional composition is dissolved in a minimal amount of water, such as about 100 to 200 ml, interruption of sleep due to an urge to urinate or due to an uncomfortable feeling of being too full may be avoided.

Accordingly, the nutritional composition is preferably a powder, a tablet, preferably a water-soluble powder or water-soluble tablet, a gel, a capsule or a pill. The nutritional composition for use according to the invention is preferably packaged in a sachet, in a strip, or in a box, more preferably as a unit dose (serving).

Alternatively, the nutritional composition may be formulated as a food product, such as a bar, a cookie, a drink, a shake, a gel, a yoghurt or the like.

### Medical Use

Advantageously, the nutritional composition according to the invention is for use as a medicament. In particular The nutritional composition can be a medicament to be administered in the form of a medical nutritional product. Thus, the nutritional composition can be used in the treatment or prevention of a medical disorder. In particular, a nutritional composition according to the invention may be used in the prevention of a medical disorder wherein a muscle parameter (such as muscle mass or a muscle function) is adversely affected or in the treatment of a person having such medical disorder.

Accordingly, the nutritional composition according to the invention is preferably for use in a method of treatment by therapy, preferably to treat or prevent a medical condition or disease that benefits from maintaining or increasing muscle mass, muscle function or muscle strength, reducing decline of muscle mass, muscle function or muscle strength, in particular for preserving muscle mass during sleep. Therefore, the invention relates to the nutritional composition for use in a method for treatment by therapy, comprising maintaining or increasing muscle mass and/or maintaining or increasing a muscle function and/or reducing loss of muscle mass and/or reducing loss of a muscle function of a human in need thereof. In particular, good results have been achieved in improving muscle strength.

Muscle mass or loss of muscle mass may be determined using any suitable method in the art, for example by calculating the percentage of muscle mass from an MRI scan or by measuring the circumference of a muscle, e.g. the calf and compare the obtained value with a reference value. Said reference value may be an internal reference value, i.e. a value obtained at an earlier time point for the same subject or an external value, i.e. the average value for comparable subjects, e.g. humans of similar age, sex and height.

Muscle function or loss of muscle function may be determined by testing one or more parameters and comparing those with a reference value, which may be an internal reference (e.g. a value obtained for the same subject obtained at an earlier time point) or an external reference, i.e. the average value for subjects of the same sex and age. For example, the tests summarized by EWGSOP2 in Table 1 (Crutz-Jentoft, et al. 2019. Age and Ageing, 48: 16-31) may be used to estimate (loss of) muscle function in a subject.

Muscle strength or loss of muscle strength may be determined by lifting or moving a weight by muscle force of a subject and comparing the total weight lifted or moved with a reference value (which may be internal or external, as substantiated herein). An example of a suitable way of measuring muscle strength is measuring hand grip strength with a hand dynamometer.

The nutritional composition for use according to the invention may be administered to any human that suffers from or is at risk of reducing muscle mass, experiencing a reduce muscle function (e.g. strength) or both. Therefore, the nutritional composition according to the invention is preferably for use in the treatment of any medical condition that is associated with loss of muscle mass, loss of muscle function or loss of muscle strength.

Examples of such medical conditions include sarcopenia, cachexia, including cancer cachexia and anorexia.

Preferably, the nutritional composition according to the invention is for use in the treatment or prevention of sarcopenia. As substantiated herein above, the nutritional composition according to the invention comprises components that are beneficial in promoting muscle anabolism and inhibiting muscle catabolism. These processes are advantageous in treating or preventing sarcopenia, as substantiated herein above.

Persons that can benefit from consuming a nutritional composition in accordance with the invention can be selected from any age group, in particular any adult age group, including humans in an age group selected from 18-30 years, 30-45 years, 45-65 years, 65-80 years, 80-100 years and 100+ years.

According to the invention, the nutritional composition is further for use in humans, preferably adults (humans of the age of 18 years or older), that are physically inactive, for example, because they are recovering from an injury or surgery. Alternatively or additionally, said nutritional composition may be for use in humans suffering from a disease or condition that makes the subject physically inactive or prevents the subject to consume sufficient amounts of nutrients. For example, as a result of generally feeling unwell, feeling exhausted or of suffering from nausea, thereby being unable to consume sufficient nutrients. Examples of such medical conditions are for example HIV/AIDS, cancer or nausea. In such cases, the nutritional composition according to the invention may beneficially support muscle anabolism, thereby preventing or reducing loss of muscle mass, loss of muscle function and/or loss of muscle strength.

The nutritional composition according to the invention is therefor also particularly suitable for use in treatment of physically inactive subjects, such as hospitalized and nursing home subjects.

In an advantageous embodiment, the nutritional composition according to the invention is for use in treatment of women, in particular for use in maintaining or increasing muscle mass, muscle function or muscle strength, reducing decline of muscle mass, muscle function or muscle strength in women, preferably elderly women. More specifically, the nutritional composition according to the invention is for use in treating women suffering from or at risk of developing sarcopenia.

The nutritional composition for use in a method of treatment by therapy, preferably comprising reducing loss of muscle mass and/or reducing loss of muscle function and/or maintaining or increasing muscle mass and/or maintaining or increasing muscle function of a human in need thereof, is preferably administered as a serving of about 80 to about 400 kcal, more preferably a serving of about 120 to about 200 kcal, in particular a serving of about 150 to about 200 kcal, such as a serving of about 180 kcal or about 190 kcal. Preferably, said nutritional composition is administered orally. In particular a serving of the nutritional composition according to the invention is administered as a food product intended for oral use, preferably a bar, a cookie, a drink, a shake, a gel (e.g. gummies or a gelled dessert) or a yoghurt. In an advantageous embodiment, the nutritional composition is provided to the intended consumer of the composition as a powder. The consumer can then decide to e.g. reconstitute the product in a volume of water or an in another aqueous fluid, as desired, usually in a volume of about 50 to 200 ml per serving. A relatively low volume, such as a volume of 150 ml or less, in particular of about 125 ml or less, is recommended especially when consumed shortly before going to sleep, to avoid urgency to urinate during sleeping time (the night), which may disrupt sleep. Besides its effect on sleep quality, it is considered by the inventors that at least in some embodiments, avoiding sleep disruption can positively contribute to protein synthesis in accordance with the invention.

The nutritional composition for use according to the invention, is preferably administered prior to going to sleep, more preferably about 1 hour or less, about 45 minutes or less, about 30 minutes or less, in particular about 15 minutes or less, prior to going to sleep. As substantiated herein above, proteinaceous matter ingested before sleep was properly digested and absorbed throughout the night. Hence, by administering the nutritional composition prior to sleep, the levels of amino acids provided by the nutritional composition are relatively high during sleep, thereby supporting muscle anabolism and improving sleep quality.

Further, by administering the nutritional composition according to the invention prior to sleep, the daily caloric intake, in particular the daily intake of protein, of a subject in need thereof, in particular a subject suffering from or at risk of developing sarcopenia, may be increased.

Although current recommendations often advise against administration of food less than 1 h prior to sleep, as most food products suffer from poor digestion during sleep, it was realized that the nutritional composition according to the invention is well metabolized by the body during sleep, without negatively affecting sleep quality.

In addition, studies support that a nutritional composition consumed before sleep does not negatively affect the feeling of satiety in the morning.

Therefore, administration of a nutritional composition prior to sleep, may boost the total daily caloric intake, and in particular the daily intake of protein, of a subject in need thereof, in particular a subject suffering from or at risk of developing sarcopenia.

In particular the nutritional composition for use according to the invention is administered as a serving of about 80 to about 400 kcal, more preferably a serving of about 120 to about 200 kcal prior to going to sleep, more preferably 1 hour or less, 45 minutes or less, 30 minutes or less, in particular 15 minutes or less, prior to going to sleep. Such a nutritional composition provides sufficient nutrients required to support muscle anabolism during sleep, whilst not having a negative effect on sleep, due to the serving size being too large.

Further, the nutritional composition for use according to the invention is preferably administered to a human that has performed physical exercise less than 3 hours, more preferably less than 2 hours, in particular less than 1 hour prior to sleep. The type and intensity of the exercise will depend on the health and age of the subject. Preferably, high intensity resistance exercise is needed. This normally happens by repetitive weightlifting. However, using your own body weight is a very often used method in elderly people. Repeating getting up from your chair (chair standing) and walking stairs are examples of using your own body weight for resistance exercise. An exercise protocol should preferably be designed in collaboration with a health care professional and adapted to the subject's capacity.

In this context, physical exercise refers to any physical activity stimulating a muscle to contract compared to the resting state. Preferably, said physical exercise is resistance exercise, i.e. working muscles against an external resistance such as a weight, thereby stimulating the muscles to contract. In particular, said physical exercise comprises at least 5 minutes of physical exercise, preferably at least 10 minutes, in particular at least 15 minutes of physical exercise. In such cases, the human simultaneously benefits from the anabolic trigger provided by the physical exercise and from the nutritional composition, which triggers may mutually reinforce one another.

Preferably, the nutritional composition for use according to the invention, is administered to an elderly human, preferably above 65 years of age. As substantiated herein above, muscle loss is a significant problem in a large group of elderly humans. In a specific embodiment, the nutritional composition for use according to the invention, is administered to male humans, preferably elderly male subjects.

The invention further relates to a method of treatment, comprising administering an effect amount of the nutritional composition according to the invention to a subject in need thereof, preferably to a human in need thereof. The administration is typically via the gastro-intestinal tract, preferably orally. In a specific embodiment, the composition is tube-fed.

The invention further relates to a use of the nutritional composition according to the invention in the manufacture of a medicine for use in the treatment of loss of muscle mass and/or loss of muscle function and/or loss of muscle strength, in particular for use in the treatment of sarcopenia.

Further it has been found that a nutritional composition according to the invention can have a positive effect on sleeping behaviour, in particular sleep quality. As discussed above, and illustrated in the examples, this can contribute to an improvement in relation to a muscle parameter, e.g. in the prevention or treatment of sarcopenia. It is further contemplated that the invention is therefore useful in the treatment of a (medical) sleeping disorder.

Further, the nutritional composition according to the invention can be used to increase the daily proteinaceous matter uptake by persons or to maintain the daily proteinaceous matter uptake at a recommended level, in particular by persons whose daily intake is (chronically) below the daily recommended intake or that experience a poor uptake of proteinaceous matter by the body. Ill people that can benefit from treatment in accordance with the invention include people that experience insufficient proteinaceous matter uptake (such as insufficient to preserve or increase muscle mass or a muscle function) directly due to the nature of the illness (various muscular disorders, e.g. sarcopenia) or as a consequence of lack of sufficient physical activity (as a consequence of the illness, e.g. due to being bedridden).

A recommended (average) daily proteinaceous matter intake is generally in the range of about 0.8 to about 1.0 g/kg body weight for healthy adults, typically about 0.8 g/kg for relatively young adults (in particular of less than 50 years of age or less) respectively about 1.0 g/kg body weight for relatively old adults (in particular of 50 years of age or more, more in particular of 65 years of age or more).

When referring herein to an average daily intake, this means generally an average per 7 days or less, preferably per 3 days, more preferably per 2 days. When used in the treatment of an ill person, in particular an ill person experiencing muscle mass loss or muscle function loss or being at risk of losing muscle mass or muscle function, the composition according to the invention is advantageously used for increasing the (average) daily proteinaceous matter intake to a value of 1.0 g/kg body weight or more, in particular in the range of 1.2-1.5 g/kg body weight or to maintain the (average) daily proteinaceous matter intake at a value of 1.0 g/kg body weight or more, in particular of 1.2-1.5 g/kg body weight. In particular elderly ill persons benefit from an (average) daily proteinaceous protein intake of at least 1.2 g/kg body weight.

Insufficient proteinaceous matter intake is in particular a potential medial issue for many elderly people, ill people (chronically ill or suffering from an acute illness), people recovering from injury or surgery and chronically malnourished people in general. The composition can adequately be consumed, e.g. as an easy to drink liquid, in a small volume, without replacing any meals (fully or substantial parts thereof) of a person's normal eating pattern. Thus, the composition can advantageously be administered supplement a person's diet. I.e. it can be consumed without any further substantial changes in the diet.

In particular, the composition can be consumed more than an hour after the last meal of the day, shortly before going to sleep (such as within an hour or within 30 min before going to sleep). Thus, it is possible to increase the daily intake of proteinaceous matter by people in need thereof considerably, preferably with at least about 15%, in particular with at least about 20%, more in particular with 25-40%.

### Non-Medical Use

The invention further relates to a non-medical use of the nutritional composition according to the invention in preserving muscle mass during sleep. For example, the nutritional composition may be administered for cosmetic or nutritional purposes. Hence, the nutritional composition may, in accordance with the invention, be administered to athletes aiming to increase or maintain muscle mass, or muscle strength, or to individuals that wish to increase muscle mass for aesthetic purposes, e.g. to modify the shape of the body.

In particular, the nutritional composition according to the invention may be administered to healthy subjects, preferably healthy humans, which may be 65+ years of age or which may be healthy humans having an age of 65 years or less, 60 years or less, 55 years or less, or 50 years or less.

Alternatively, the nutritional composition according to the invention may be administered to an elderly mammal that does not suffer from sarcopenia.

As mentioned above the nutritional composition according to the invention can be used to increase the daily proteinaceous matter uptake by persons. In an embodiment, the composition is used non-medically for increasing daily proteinaceous matter intake. In this embodiment, the composition can in particular be a sports nutrition or a healthy aging nutrition. When used non-medically, the composition according to the invention is typically administered in an effective amount to obtain a(n average) daily proteinaceous matter intake in the range of about 0.8-1.0 kg/kg body weight.

As illustrated in the examples, the nutritional composition has also been found suitable to improve sleep quality as determined by the Pittsburgh Sleep Quality Index. This effect can be beneficial for medical reasons. However, such effect can also be beneficial for non-medical reasons, e.g. for humans not experiencing pathological effects due to sleeping problems, but experiencing a (temporary) inconvenience in sleeping quality, e.g. due to travelling through multiple time zones in a short time (jet-lag related loss of sleeping quality), parenting issues, or irregular working times. Thus, in an embodiment, the invention relates to the (non-medical) use of a nutritional composition according to the invention to improve sleep quality as determined by the Pittsburgh Sleep Quality.

For the purpose of clarity and a concise description, features are described herein as part of the same or separate embodiments, however, it will be appreciated that the scope of the invention may include embodiments having combinations of all or some of the features described.

The invention is demonstrated by the following examples.

### EXAMPLES 1-10: FORMULATIONS OF NUTRITIONAL COMPOSITIONS ACCORDING TO THE INVENTION

Examples of nutritional compositions according to the invention are described herein below, which are particularly suitable to be used as a single serving (to be consumed once a day, preferably shortly before sleep).

Example 1:
2.5 Gr Casein protein
12.5 Gr Whey protein
400 mg free L-serine or salt thereof
2.2 Gr free glycine or salt thereof
Example 2:
7.5 Gr Casein protein
7.5 Gr Whey protein
2.2 Gr free glycine or salt thereof
500 mg free L-serine or salt thereof
Example 3:
15 Gr Milk protein
1.5 Gr HMB
400 mg free L-serine or salt thereof
2.2 Gr free glycine or salt thereof
Example 4:
15 Gr milk protein
3 Gr Almond protein
1.5 Gr HMB
2.2 Gr free glycine or salt thereof
500 mg free L-serine or salt thereof
Example 5:
15 Gr milk protein
1.5 Gr free form HMB
2.2 Gr free glycine or salt thereof
500 mg free L-serine or salt thereof
200 mg Choline
200 mg EPA/DHA
Example 6:
7.5 Gr Casein protein
7.5 Gr Whey protein
5.0 Gr Soy protein
1.5 Gr HMB
2.5 Gr of total glycine (typically around 2 gr free glycine or salt thereof)
8% of total proteinaceous matter L-serine (typically around 600 mg free
L-serine or salt thereof)
200 mg Choline
200 mg EPA/DHA
Total product 200 kcal
Example 7:
7.5 Gr Casein protein
7.5 Gr Whey protein
5.0 Gr Soy protein
1.5 Gr HMB
2.5 Gr of total glycine (typically around 2 gr free glycine or salt thereof)
8% of total proteinaceous matter L-serine (typically around 600 mg free
L-serine or salt thereof)
200 mg Choline
200 mg EPA/DHA
Total product 200 kcal
Example 8:
7.5 Gr Casein protein
7.5 Gr Whey protein
5.0 Gr Soy protein
1.5 Gr HMB
2.5 Gr of total glycine (typically around 2 gr free glycine or salt thereof)
8% of total proteinaceous matter L-serine (typically around 600 mg free
L-serine or salt thereof)
200 mg Choline
200 mg EPA/DHA
Vitamin/mineral mixture at 10% DRI
Total product 180 kcal
Example 9:
7.5 Gr Casein protein
7.5 Gr Whey protein
3.0 Gr Almond protein
1.5 Gr HMB
2.5 Gr of total glycine (typically around 2 gr free glycine or salt thereof)
8% of total proteinaceous matter L-serine (typically around 600 mg free
L-serine or salt thereof)
200 mg Choline
200 mg EPA/DHA
Vitamin/mineral mixture at 10% DRI
Total product 200 kcal
Example 10:
about 2.8 Gr Casein protein
about 15.4 Gr Whey protein
about 2.2 Gr soy protein
about 1.5 Gr calcium HMB
about 2.5 Gr of total glycine (of which about 2 gr free glycine or salt thereof)
8% of total proteinaceous matter L-serine (of which about 715 mg free
L-serine or salt thereof)
about 200 mg Choline
Fat, preferably up to about 2 gr
Carbohydrate, preferably up to about 21 gr
Optionally vitamin/mineral mixture at up to 10% DRI
Total product about 200 kcal

### EXAMPLE 11

Four adult humans of diverse age were enrolled in this pilot study. All except one had sleeping complaints.

At baseline, before start of treatment and after 1 month of treatment, the sleep quality was measured by the Pittsburgh Sleep Quality Index (PSQI) which is a generally known standardized sleep questionnaire for clinicians and researchers (described in Buysse D. J. et al. (1988) The Pittsburgh Sleep Quality Index: A new instrument for psychiatric practice and research. Psychiatry Research 28, 193-213). It is a self-rated questionnaire assessing sleep over a 1-month time interval. It is a 7-components score looking into the different aspects of sleep quality with the maximum score of 21 (very bad sleep quality) and the minimal score of 0 (very good sleep quality).

Further, at baseline and after 1 month, the handgrip strength (kg) was measured with an electronic hand dynamometer (model Camry EH101). This served as a measure of muscle strength.

The test product was made by physically mixing the ingredients (casein, WPC80, soy protein, glycine in a free form, serine in a free form, Ca HMB, choline (as bitartrate), flavour), to obtain the test product in a powder form. The obtained mixture had the following characteristics:

| | | % of |
|---|---|---|
| Test product (powder) | | proteinaceous |
| (50 gram per serving): | gr/100 kcal | matter |
| 2.8 Gr Casein protein*# | 1.4 | 12.1 |
| 15.4 Gr Whey protein*# | 7.9 | 66.3 |
| 2.2 Gr Soy protein# | 1.1 | 9.5 |
| 2.5 Gr of total glycine (of | 1.3 | 10.8 |
| which 2.1 gr free | (of which 1.1 g/100 kcal | (of which |
| glycine#) | free glycine) | 9.0 free) |
| 8% of total protein Serine | 0.95 | 8 |
| (of which 715 mg free L- | (of which 365 mg/100 kcal | (of which 3.1% |
| serine#) | free L-serine) | free L-serine) |
| 1.8 Gr Fat** | 0.9 | n.a. |
| 18.6 Gr Carbohydrates** | 10.3 | n.a. |
| 1.5 Gr calcium HMB | 0.76 | n.a. |
| 200 mg Choline^{$} | 102 | n.a. |
| minor components of | balance | balance |
| products ingredients | | |
| (such as salts, dietary | | |
| fibre, moisture, counter | | |
| ions) and further flavour | | |
| Total product 196 kcal | | |

*total milk protein: 9.9 g/100kcal (78 wt. % of proteinaceous matter)
**source: from protein ingredients (fat, carbohydrate), and HMB (carbohydrate)
#total proteinaceous matter: 12.7 g/100 kcal
^{$}included as choline bitartrate (490 mg)
n.a.: not applicable

After the baseline measurement, the subjects daily reconstituted 50 gr of the test product in a volume of tap water (typically 100-150 ml) and daily consumed the reconstituted product for 1 month before going to sleep (30 min-1 hour).

The results of the sleep quality and handgrip strength before and after 1 month supplementation of the product are shown in in FIGS. 2 and 3 respectively. It is in particular remarkable that after only 1 month of consuming the product already an effect on an improvement of a muscle parameter (muscle strength) was observed. It should be noted that the protein component of the product was not supplemented with additional free branched amino acids, such as leucine, which are usually considered to be useful to stimulate muscle synthesis. The product did contain a higher serine content than known products often consumed in the evening (such as milk-based desserts, milk). Also the glycine content was relatively high. Without being bound to theory these data support the considerations made in the present description, such as the considerations regarding a correlation between sleeping quality and preservation of a muscle parameter or even improvement thereof, notably muscle function.

1. A nutritional composition, comprising, about 5 to 25.0 g per 100 kcal of proteinaceous matter, of which about 5 to 24.9 g per 100 kcal of proteinaceous matter is derived from milk; wherein
the serine content of the nutritional composition is at least about 7 wt. %, based on the weight of the proteinaceous matter; and wherein
the glycine content of the nutritional composition is between 5 and 30 wt. % based on the weight of the proteinaceous matter.

2. (canceled)

3. The nutritional composition according to claim 1, comprising:
(a) about 150 to about 300 mg per 100 kcal of free serine or a salt thereof;
(b) about 300 to about 600 mg per 100 kcal of free serine or a salt thereof; and/or
(c) about 300 mg per 100 kcal to about 2000 mg per 100 kcal of free glycine or a salt thereof.

4-5. (canceled)

6. The nutritional composition according to claim 1, wherein the total content of glycine and serine is at least 1.5 g per 100 kcal.

7. The nutritional composition according to claim 1, further comprising:
(a) about 400 mg per 100 kcal to about 2500 mg per 100 kcal of beta-hydroxy beta-methylbutyric acid (HMB); and/or
(b) about 50 to about 150 mg of an omega-3-fatty acid selected from eicosapentaenoic acid (EPA), docosahexaenoic acid (DHA) and a combination thereof.

8. (canceled)

9. The nutritional composition according to claim 1, comprising about 5.5 to about 20 g per 100 kcal proteinaceous matter derived from milk.

10. The nutritional composition according to claim 7, comprising, about 7 to about 15 g per 100 kcal of proteinaceous matter, of which about 5 to about 10 g per 100 kcal of proteinaceous matter derived from milk;
about 750 to about 1250 mg per 100 kcal of free glycine or a salt thereof; and
about 200 to about 300 mg per 100 kcal of free serine or a salt thereof;
about 600 to about 900 mg per 100 kcal of free beta-hydroxy beta-methylbutyric acid (HMB);
about 75 to about 125 mg per 100 kcal of an omega-3-fatty acid selected from eicosapentaenoic acid (EPA), docosahexaenoic acid (DPA) and a combination thereof; and further comprising:
about 75 to about 125 mg per 100 kcal of choline; and
carbohydrates and/or fat.

11. The nutritional composition according to claim 1, wherein the composition comprises
60 to 90 wt. % milk protein selected from the group consisting of casein and whey protein,
2.6-15 wt. % free serine or a salt thereof,
5-15 wt. % free glycine or a salt thereof,
all based on weight of the proteinaceous matter.

12. The nutritional composition according to claim 1, comprising:
(a) 3.0-10 wt. % free serine or a salt thereof; and/or
(b) 8.0-12 wt. % free glycine or a salt thereof.

13. (canceled)

14. The nutritional composition according to claim 10, comprising 500 to 1500 mg/100 kcal calcium HIB and about 30 mg to about 150 mg/100 kcal choline.

15. The nutritional composition according to claim 1, wherein the nutritional composition comprises milk fat.

16. The nutritional composition according to claim 1, packaged as a serving of about 80 to about 400 kcal.

17. The nutritional composition according to claim 1, wherein the nutritional composition is a powder, a tablet, a capsule, a pill or a food product selected from the group consisting of a bar, a cookie, a drink, a shake, a gel, and a yoghurt.

18. (canceled)

19. A method of treatment by therapy of a subject in need thereof, the method comprising administering to the subject in need thereof the nutritional composition according to claim 1.

20. (canceled)

21. The method according to claim 19, wherein the therapy comprises maintaining or increasing muscle mass and/or maintaining or increasing muscle function of a human in need thereof.

22. A method of treating or preventing sarcopenia in a subject in need thereof, the method comprising administering to the subject in need thereof the nutritional composition according to claim 1.

23. The method according to claim 19, wherein the nutritional composition is administered to a human above 50 years of age.

24. The method according to claim 19, wherein the nutritional composition is administered to a human as a serving of about 80 to about 400 kcal, wherein the serving is administered 1 hour or less before sleep.

25. The nutritional composition for use according to claim 24, wherein the human has performed physical exercise less than 3 hours prior to sleep.

26. A method of treating a sleeping disorder in a subject in need thereof, the method comprising administering to the subject in need thereof the nutritional composition according to claim 1.

27. A method for improving sleep quality as determining by the Pittsburgh Sleep Quality Index in a subject in need thereof, the method comprising administering to the subject in need thereof the nutritional composition according to claim 1.

28. The method of claim 19, wherein the therapy comprises increasing the average daily proteinaceous matter intake of a person in need thereof to a value of at least 0.8 g/kg bodyweight or maintain at a value of at least 0.8 g/kg bodyweight.

29. The method according to claim 28, wherein the subject is a malnourished human and the value is increased to or kept at a value in the range of 1.2-1.5.

30. The method according to claim 28, wherein the subject has an illness, which illness may be chronically or acute.

31. A method for increasing the average daily proteinaceous matter intake of a person in need thereof to a value of about 0.8-1.0 g/kg bodyweight, the method comprising administering to the person in need thereof the nutritional composition according to claim 1.

32. The method according to claim 31, wherein the nutritional composition is used as a sports nutrition or healthy aging nutrition.

33. The method Use-according to claim 31, wherein the nutritional composition is administered to a human as a serving of about 80 to about 400 kcal, wherein the serving is administered to the human 1 hour or less before sleep.

34. The method according to claim 33, wherein the human has performed physical exercise less than 3 hours prior to sleep.
